(19) United States
(12) Patent Application Publication
(10) Pub. No.: US 2009/0253913 A1
(43) Pub. Date: Oct. 8, 2009

(54) Process for the preparation of sorafenib and salts thereof

(76) Inventors: Pierluigi Rossetto, Lodi (IT); Peter Lindsay MacDonald, Gentilino (CH); Augusto Canavesi, Locate Varesino (CO) (IT)

Correspondence Address: KENYON & KENYON LLP, ONE BROADWAY, NEW YORK, NY, 10004, US

(21) Appl. No.: 12/381,000

(22) Filed: Mar. 5, 2009

Related U.S. Application Data
(60) Provisional application No. 61/068,478, filed on Mar. 6, 2008.
(60) Provisional application No. 61/150,169, filed on Feb. 5, 2009.

(51) Int. Cl. C07D 213/78; C07D 233/14

(52) U.S. Cl. 546/298; 548/334.1

(57) ABSTRACT

Methods for the synthesis of the N-carbamoyl imidazole (I) and its 1:1 adduct with imidazole are provided. Methods for the preparation of these crystalline intermediates in a high state of purity are also provided. These intermediates react cleanly under mild conditions to produce sorafenib in high yield and purity, without generating difficult-to-remove impurities.

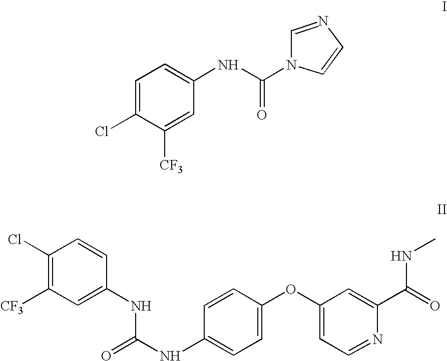

### RELATED APPLICATIONS

This application claims priority of U.S. Provisional Application No. 61/068,478, filed Mar. 6, 2008 and U.S. Provisional Application No. 61/150,169, filed Feb. 5, 2009, the entire contents of which are incorporated by reference herein.

### FIELD OF INVENTION

The present invention relates to intermediates of sorafenib base, their preparation and conversion to sorafenib base and salts thereof.

### BACKGROUND

Sorafenib base, 4-[4-[[4-chloro-3-(trifluoromethyl)phenyl]carbamoylamino]phenoxy]-N-methyl-pyridine-2-carboxamide of the following formula:

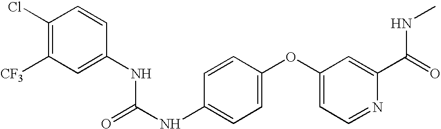

is an intermediate for sorafenib salts, such as sorafenib tosylate of the following formula.

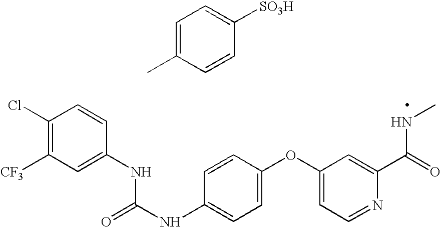

Sorafenib tosylate is marketed as Nexavar® by Bayer for the treatment of advanced renal cell carcinoma. It is a small molecular inhibitor of Raf kinase, PDGF (platelet-derived growth factor), VEGF (vascular endothelial growth factor) receptor 2 and 3 kinases, and c-Kit (the cytokine receptor for stem cell factor).

U.S. Pat. No. 7,235,576 discloses processes for preparation of sorafenib base and analogues thereof.

According to one process, N-(2-methoxy-5-(trifluoromethyl)phenyl)-N′-(4-(2-(N-methylcarbamoyl)-4-pyridyloxy)phenyl)urea, which is a dechlorinated analogue of sorafenib, is prepared by coupling 4-(2-(N-methylcarbamoyl)-4-pyridyloxy)aniline of formula 2, 5-(trifluoromethyl)-2-methoxyaniline and 1,1′-carbonyldiimidazole (“CDI”), as illustrated by the following scheme:

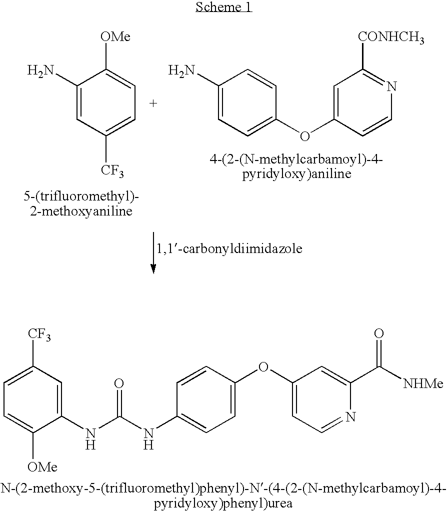

and purifying the obtained product by column chromatography. This patent also reports that the reaction of an aniline derivative, such as 4-(2-(N-methylcarbamoyl)-4-pyridyloxy)aniline of formula 2, with 1,1′-carbonyldiimidazole (“CDI”), leads also to the formation of symmetrical urea impurities, such as 1,3-bis-[4-(2-(N-methylcarbamoyl)-4-pyridyloxy)phenyl]urea of the following formula:

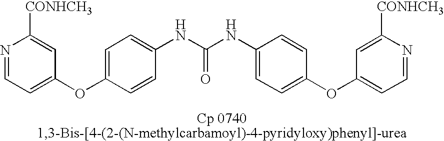

without describing how to purify from them.

U.S. Pat. No. 7,235,576 and WO 2006/034796 also report a process for preparation of sorafenib base, which is obtained as an off-white solid in U.S. Pat. No. 7,235,576 or as colorless to slightly brownish crystals in WO 2006/034796. In this process, sorafenib is prepared by coupling between 4-(2-(N-methylcarbamoyl)-4-pyridyloxy)aniline of formula 2 and 4-chloro-3-(trifluoromethyl)phenyl isocyanate of formula 1, as illustrated by the following scheme:

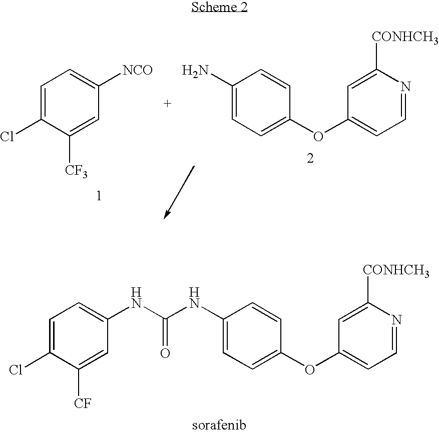

The isocyanate of formula 1, which is a reactive derivative of carbonic acid, can be prepared, according to U.S. Pat. No. 2,745,874, from its corresponding aniline derivative. This patent also describes the reaction between the isocyanate and an aminobenzene compound, such as aniline, to provide derivatives of diphenyl urea such as 1,3-Bis-(4-choloro-3-trifluoromethyl-phenyl)-urea of the following formula:

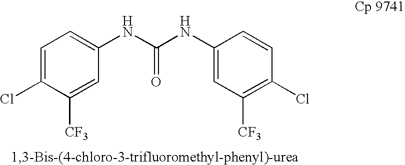

Bankston et al. (Organic Process Research & Development 2002 Vol. 6 p. 777-781) relates to the two processes in scheme 1 and 2, and to the urea impurities that are formed in them.

Thus, there is a need in the art for a new process that provides sorafenib in high yield and purity.

### SUMMARY OF INVENTION

In one embodiment, the present invention encompasses the compound of formula 4 of the following structure;

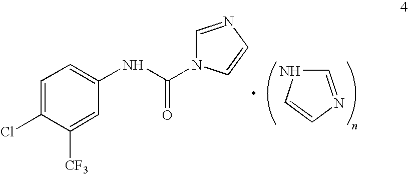

wherein n is either 0 or 1.

In another embodiment the present invention provides the use of the compound formula 4:

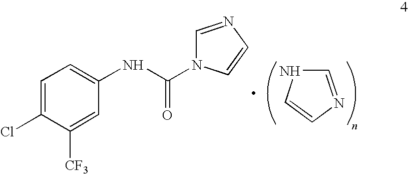

to prepare sorafenib and salts thereof of the following formula:

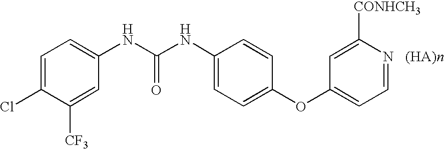

wherein n is either 1 or 0, and HA is an acid, preferably p-toluenesulfonic acid (“PTSA”).

In another embodiment, the present invention encompasses a process for preparing sorafenib base comprising reacting the compound of formula 4:

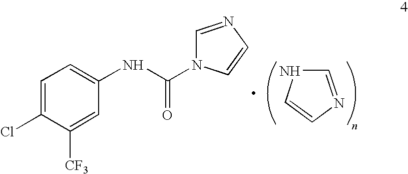

with 4-(2-(N-methylcarbamoyl)-4-pyridyloxy)aniline of formula 2:

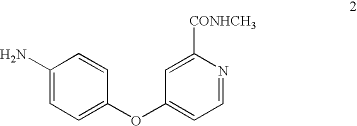

to obtain sorafenib base, wherein n is either 1 or 0.

In yet another embodiment, the present invention encompasses a process for preparing sorafenib salt comprising preparing sorafenib base according to a process of the present invention, and converting it to sorafenib salt. Preferably, the salt is PTSA salt.

In one embodiment, the present invention encompasses a process for preparing imidazole complex of imidazole-1-carboxylic acid 4-chloro-3-(trifluoromethyl)phenyl amide of formula 4a of the following structure:

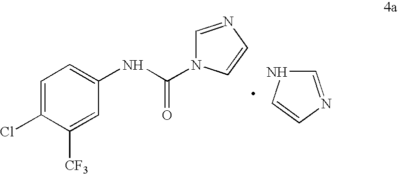

comprising reacting 4-chloro-3-(trifluoromethyl)aniline of the following formula 3:

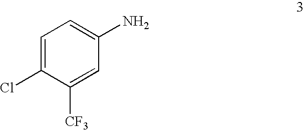

with carbonyldiimidazole (CDI) of formula 5 having the following structure;

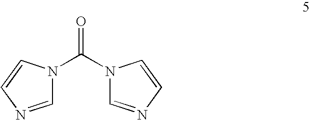

to obtain an imidazole complex of imidazole-1-carboxylic acid 4-chloro-3-(trifluoromethyl)phenyl amide of formula 4a.

In another embodiment, the present invention encompasses a process for preparing imidazole-1-carboxylic acid 4-chloro-3-(trifluoromethyl)phenyl amide of formula 4b of the following structure:

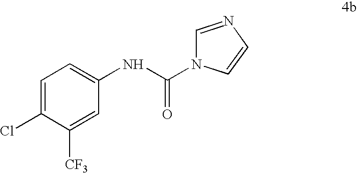

comprising reacting 4-chloro-3-(trifluoromethyl)phenyl isocyanate of formula 1:

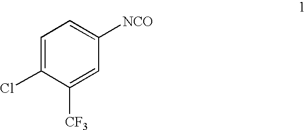

with imidazole or a salt thereof of formula 6:

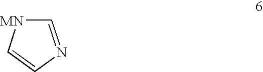

wherein M is hydrogen, sodium or potassium;
to obtain imidazole-1-carboxylic acid 4-chloro-3-(trifluoromethyl)phenyl amide of formula 4b.

In another embodiment, the present invention encompasses a process for preparing sorafenib and salt thereof comprising preparing the compounds of formula 4a or 4b according to a processes of the present invention, and converting either of them to sorafenib and salt thereof.

### DETAILED DESCRIPTION OF THE INVENTION

The present invention relates to intermediates of sorafenib base, their preparation and conversion to sorafenib base and salts thereof.

The processes of the prior art lead directly to the formation of sorafenib without an intermediate step. Hence, the urea impurities which are formed during the process are not removed and thus contaminate sorafenib. Sorafenib is relatively not soluble in many solvents and thus purifying it is a challenge, i.e., trials to reduce the levels of impurities such as those mentioned In the prior art were unsuccessful, thus provided sorafenib contaminated with about 10% of urea impurities.

The present invention offers a process via two novel intermediates that are isolated. The isolation of these intermediates allows to purify the intermediate, especially from the urea impurities, thus leading to highly pure sorafenib in high yields that is obtained without any additional purification, but by simply precipitating it from the reaction mixture. Since sorafenib can be produced by this process in high yields and purity avoiding time consuming purification processes, this process is advantageous for industrial scale.

A skilled in the art would appreciate that the process of the present invention can also be adapted for the preparation of other aryl urea derivatives in high yield and purity.

In one embodiment, the present invention encompasses the compound of formula 4 of the following structure:

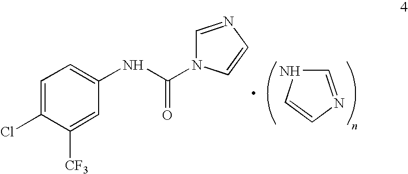

wherein n is either 0 or 1. Preferably, n is 1.

In a preferred embodiment, above compound of formula 4 is provided in an isolated form. Preferably, the isolated compound of formula 4 is solid, more preferably it is crystalline.

As used herein, the term isolated in reference to the compound of formula 4 corresponds to a compound of formula 4 that is physically separated from the reaction mixture, where it is formed. For example, the separation can be done by filtering the precipitated compound of formula 4. More preferably, the compound of formula 4 is separated from 1,3-bis-(4-choloro-3-trifluoromethyl-phenyl)urea. The presence of 1,3-bis-(4-choloro-3-trifluoromethyl-phenyl)urea can be detected by TLC.

When n is 1, the compound of formula 4 is an imidazole complex of imidazole-1-carboxylic acid 4-chloro-3-(trifluoromethyl)phenyl amide of formula 4a, having the following structure:

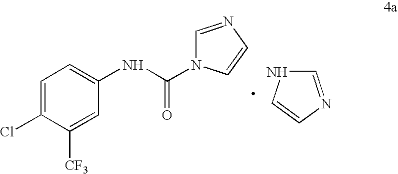

The isolated an imidazole complex of imidazole-1-carboxylic acid 4-chloro-3-(trifluoromethyl)phenyl amide of formula 4a can be used as an advantageous intermediate in the preparation of sorafenib, due to the removal of excess of carbonyldiimidazole (CDI) when isolating the compound of formula 4a. If CDI is not removed, it reacts with 4-(2-(N-methylcarbamoyl)-4-pyridyloxy)aniline of formula 2 in the process for preparing Sorafenib base, to further provide 1,3-bis-[4-(2-(N-methylcarbamoyl)-4-pyridyloxy)phenyl]urea, which contaminates Sorafenib and is difficult to purify from.

The above compound of formula 4a can be characterized by a ¹H-NMR (300 MHz) spectrum in CDCl₃ having chemical shifts at 7.07, 7.12, 7.46, 7.67, 7.77, 7.83, 7.92, 8.45 and 10.7 ppm. When n is 0, the compound of formula 4 is imidazole-1-carboxylic acid (4-chloro-3-trifluoromethylphenyl) amide of formula 4b, having the following structure:

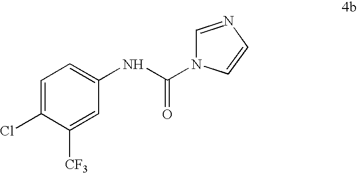

The above compound of formula 4b can be characterized by data selected from the group consisting of: a ¹H-NMR (300 MHz) spectrum in CDCl₃ having chemical shifts at about 7.913, 7.836, 7.480, 8.418, 7.725 and 7.097 ppm; MS-ESI spectrum of [M+H]⁺=m/z 290/292; and combinations thereof.

The compound of formula 4 can be used for the preparation of sorafenib and salts thereof having the following structure:

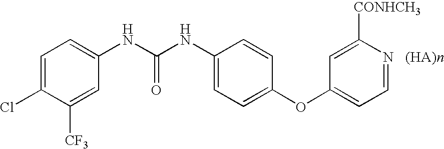

as described below, wherein n is either 0 or 1, and HA is an acid, preferably, p-toluenesulfonic acid (“PTSA”), i.e., sorafenib tosylate.

When n is 0, the above compound refers to sorafenib base, and when n is 1 the above compound refers to a sorafenib salt, preferably, sorafenib tosylate.

The process comprises reacting the compound of formula 4 of the following structure:

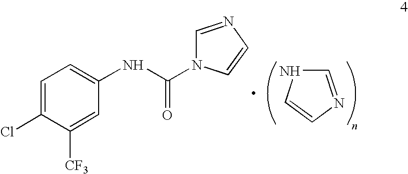

and 4-(2-(N-methylcarbamoyl)-4-pyridyloxy)aniline of formula 2 of the following structure:

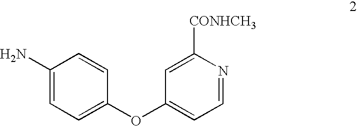

to obtain sorafenib base, wherein n is either 0 or 1.

When the starting material is the compound of formula 4a, i.e., n is 1, the imidazole complex of imidazole-1-carboxylic acid 4-chloro-3-(trifluoromethyl)phenyl amide of formula 4a can be prepared by a process comprising reacting 4-chloro-3-(trifluoromethyl) aniline of formula 3 with carbonyldiimidazole (CDI) of formula 5, as shown in the following scheme:

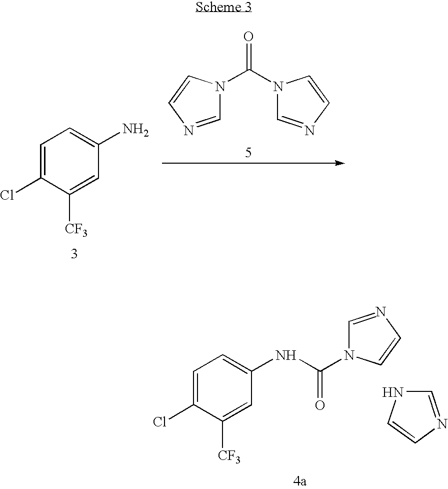

First, a solution of 4-chloro-3-(trifluoromethyl)aniline of formula 3 is provided. Preferably, the solvent is a halogenated hydrocarbon. More preferably, the halogenated hydrocarbon is a C₁-C₆ halogenated hydrocarbon, most preferably, the C₁-C₆ halogenated hydrocarbon is dichloromethane, dichloroethane or chlorobenzene.

Then, 1,1′-carbonyldiimidazole (“CDI”) of formula 5 is added to obtain a mixture.

Preferably, CDI is added in an amount of about 1 mole equivalent to about 1.2 mole equivalent per mole equivalent of 4-chloro-3-(trifluoromethyl)aniline of formula 3, more preferably about 1 mole equivalent per mole equivalent of 4-chloro-3-(trifluoromethyl)aniline of formula 3.

Preferably, the mixture is then maintained to obtain a solution. Preferably, the mixture is maintained at a temperature of about 20° C. to about 40° C., preferably 20° C. to about 35° C., more preferably 20° C. to about 30° C. Most preferably, the mixture is maintained at a temperature of about 30° C.

The solution is stirred; preferably for a period of about 8 hours to about 24 hours, more preferably 10 hours to about 20 and most preferably 16 hours to about 24, especially about 16 hours. Preferably, the stirring is done at a temperature of about 0° C. to about 30° C., more preferably at about 5° C. to about 15° C., during which time the compound of formula 4a precipitates.

The precipitated imidazole complex of imidazole-1-carboxylic acid 4-chloro-3-(trifluoromethyl)phenyl amide of formula 4a is then recovered. The recovery may be done, for example, by filtering, washing, and drying under vacuum; preferably, drying is done at a temperature of about 18° C. to about 30° C., preferably about 20° C. to about 22° C. Preferably, drying is done for a period of about 4 hours to about 16 hours, more preferably about 12 hours to about 16 hours.

When the starting material is the compound of formula 4b, i.e., n is 0, the imidazole-1-carboxylic acid 4-chloro-3-(trifluoromethyl)phenyl amide of formula 4b can be prepared by a process comprising reacting 4-chloro-3-(trifluoromethyl)phenyl isocyanate of formula 1 and imidazole or salt thereof of formula 6, wherein M is hydrogen, sodium or potassium. The process can be illustrated by the following scheme:

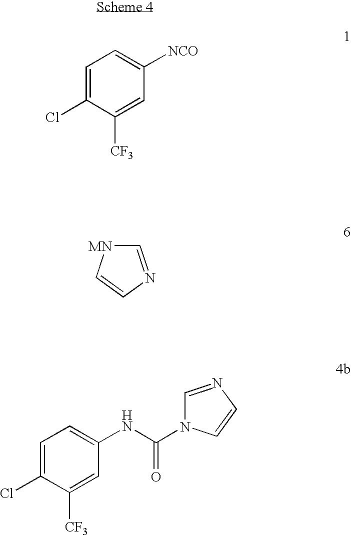

First, a solution of imidazole or a salt thereof of formula 6 is provided. M may be hydrogen, sodium, or potassium, and is preferably hydrogen. Preferably, the solvent is a halogenated hydrocarbon, preferably a chlorinated hydrocarbon. More preferably, the halogenated hydrocarbon is a C₁-C₆ halogenated hydrocarbon and preferably a C₁-C₆ chlorinated hydrocarbon, most preferably, the C₁-C₆ halogenated hydrocarbon is chloroform.

Then, 4-chloro-3-(trifluoromethyl)phenyl isocyanate of formula 1 is added to the solution.

Preferably, the amount of imidazole is about 1 mole equivalent to about 1.2 mole equivalent per mole equivalent of 4-chloro-3-(trifluoromethyl)phenyl isocyanate of formula 1, more preferably about 1 mole equivalent per mole equivalent of 4-chloro-3-(trifluoromethyl)phenyl isocyanate of formula 1.

Preferably, the solution is then maintained to obtain a suspension, thus providing a precipitate of imidazole-1-carboxylic acid 4-chloro-3-(trifluoromethyl)phenyl amide of formula 4b. Preferably the suspension is maintained at a temperature of about 15° C. to about 25° C., more preferably at a temperature of about 20° C. to about 22° C. Preferably, it is maintained for a period of about 0.5 hour to about 2 hours, more preferably for a period of about 1 hour.

Preferably the suspension is then cooled, prior to recovering imidazole-1-carboxylic acid 4-chloro-3-(trifluoromethyl)phenyl amide of formula 4b. Preferably, cooling is to a temperature of about 10° to about −5°, more preferably to about 0° C. Preferably, the suspension is further maintained at such temperature for a period of about 0.5 hour to about 2 hours, more preferably of about 1 hour.

The precipitated imidazole-1-carboxylic acid (4-chloro-3-trifluoromethyl-phenyl)-amide of formula 4b is then be recovered. The recovery may be done, for example, by filtering, washing, and drying under vacuum. Preferably, washing is done with chloroform. Preferably, drying is done at a temperature of about 35° C. to about 45° C., more preferably about 40° C. Preferably, drying is done for a period of about 12 hours to about 20 hours, more preferably 16 hours.

The recovered imidazole complex of imidazole-1-carboxylic acid 4-chloro-3-(trifluoromethyl)phenyl amide of formula 4a, and imidazole-1-carboxylic acid 4-chloro-3-(trifluoromethyl)phenyl amide of formula 4b can then be converted to sorafenib and salts thereof.

The conversion can be done by a process comprising reacting either the compound of formula 4a or the compound of formula 4b with 4-(2-(N-methylcarbamoyl)-4-pyridyloxy)aniline of formula 2 to obtain sorafenib base.

Initially, a solution of the compound of formula 4a or the compound of formula 4b is formed in a solvent selected from the group consisting of: halogenated hydrocarbons and preferably chlorinated hydrocarbins, esters, and mixtures thereof. Preferably, the halogenated hydrocarbon is a C₁-C₆ halogenated hydrocarbon and more preferably a C₁-C₆ chlorinated hydrocarbon. Preferably, the C₁-C₆ halogenated hydrocarbon is 1,2-dichloroethane, chlorobenzene or chloroform. Preferably, the ester is C₂-C₆ ester. Preferably, the C₂-C₆ ester is ethyl acetate, ethyl formate, ethyl propionate, or methyl acetate. More preferably, the C₂-C₆ ester is ethyl acetate.

The solution is preferably heated prior to the reaction with 4-(2-(N-methylcarbamoyl)-4-pyridyloxy)aniline of formula 2. Preferably, the heating is to a temperature of about 30° C. to about 80° C., depending on the solvent used. For example, when using dichloroethane, ethyl propionate or ethyl acetate, heating is to a temperature of about 60° C. to about 70° C., when using methyl acetate or ethyl formate heating is to a temperature of about 30° C. to about 50° C. and when using chlorobenzene heating is to a temperature of about 60° C. to about 70° C.

Then, 4-(2-(N-methylcarbamoyl)-4-pyridyloxy)aniline of formula 2 is added to the heated solution to obtain a mixture.

The mixture is kept, preferably under stirring, to allow the formation of sorafenib base, which precipitates. Preferably, the mixture is kept at a temperature of about 20° C. to about 80° C., more preferably about 50° C. to about 70° C. Preferably, the mixture is kept for a period of about 15 minutes to obtain a suspension comprising the said precipitate of sorafenib base.

Typically, to increase the yield of the precipitated sorafenib base, precipitation can be followed by cooling the suspension. Preferably, the cooling is to a temperature of about 30° C. to about 10° C., more preferably to a temperature of about 20° C. to about 10° C. Optionally, to increase the yield even more, the suspension can be further maintained at the above temperatures for about 1 hour to about 20 hours, preferably about 10 hours to about 18 hours and most preferably 16 hours.

The obtained sorafenib base can then be recovered. The recovery process of sorafenib base may comprise filtering off the precipitated sorafenib base, washing, and drying. Preferably, drying is done at a temperature of about 50° C. to about 60° C.; preferably for overnight.

The recovered sorafenib is obtained as a white powder having a purity of at least about 99.7% area by HPLC.

The obtained sorafenib base can then be converted to its salts. The conversion can be done by reacting sorafenib base with an acid. Preferably, the acid is p-toluenesulfonic acid and the obtained salt is the corresponding tosylate salt. The conversion to the tosylate salt can be done, for example, according to any of the processes described in WO2006/034796.

### EXAMPLES

### Experimental

### NMR:

NMR Instrument: Varian Mercury-300, Frequency: 300.07 MHz, solvent: CDCl₃
MH analysis:
Instrument: Finnigan LCQ (ion-trap)
Method: ESI, positive ion mode
Conditions: Source Voltage (kV) 3.49, Source Current (μA) 2.98, Capillary Voltage (V) 17.86, Capillary Temp (° C.): 190.5. Mobile phase 70% aqueous methanol containing 5 mM ammonium formate. Direct inlet, samples 1 mg/ml in dichloromethane.

### Example 1

### Preparation of Sorafenib Intermediate Imidazole Complex of imidazole-1-carboxylic acid 4-chloro-3-(trifluoromethyl)phenyl amide of Formula 4a

To a solution of 4-chloro-3-(trifluoromethyl)aniline of formula 3 (15 g) in 1,2-dichloroethane (150 g) under nitrogen atmosphere, CDI (12.4 g; 1.0 eq) was added. The reaction mixture was warmed and maintained at 30° C. until the suspension had dissolved, then stirred for 16 hours at 20-22° C. The precipitate was collected, rinsed with dichloroethane, and dried at 20-22° C. overnight under vacuum. Sorafenib intermediate 4a was obtained as a colorless powder (20.8 g, 76% yield).

¹H-NMR (300 MHz) spectrum in CDCl₃: chemical shifts at about 7.46, 7.83, 10.7, 8.45, 7.07, 7.77, 7.67 and 7.12.

Elemental analysis: Theoretical C, 47.01; H, 3.10; Cl, 9.91; F, 15.93; N, 19.58; O, 4.47. Experimental C, 46.7; H, 3.13; N, 19.60

### Example 2

### Preparation of Sorafenib Base

To a solution of the imidazole complex of imidazole-1-carboxylic acid 4-chloro-3-(trifluoromethyl)phenyl amide of formula 4a (8.5 g) in 1,2-dichloroethane (60 g) at 60° C., 4-(4-aminophenoxy)-N-methyl-2-pyridinecarboxamide (4.5 g) was added, and the mixture was kept under stirring at 60-65° C. for 15 minutes until a precipitate formed. The suspension was allowed to cool slowly to 20° C., and then stirred for 16 hours. The precipitate was filtered off, washed with 1,2-dichloroethane and dried at 50° C. under vacuum overnight. Sorafenib base was obtained as a colorless powder (8 g, 93% yield, 99.9% purity).

### Example 3

### Preparation of Sorafenib Intermediate imidazole-1-carboxylic acid (4-chloro-3-trifluoromethyl-phenyl)-amide of Formula 4b

4-Chloro-3-(trifluoromethyl)phenyl isocyanate (15.0 g) was added to a solution of imidazole (4.6 g) in CHCl₃ (150 g) at room temperature. The initial solution was stirred at room temperature for 1 h, obtaining a suspension which was cooled to 0° C. and kept under stirring for 1 h. The solid was filtered off, washed with CHCl₃ and dried at 40° C. under reduced pressure for 16 h, to yield 19.2 g (98%) of intermediate 4b.

¹H-NMR (300 MHz) spectrum in CDCl₃: chemical shifts at about 7.913 (1H, d, J=2.7), 7.836 (1H, dd, J=8.7, 2.7 Hz), 7.480 (1H, d, J=8.7 Hz), 8.418 (1H, dd, J=1.4, 0.9 Hz), 7.725 (1H, dd, J=1.6, 1.4 Hz), 7.097 (1H, dd, J=1.6, 0.9 Hz).

MS-ESI spectrum: [M+H]⁺ m/z 290/292

### Example 4

### Preparation of Sorafenib Base

To a solution of imidazole-1-carboxylic acid 4-chloro-3-(trifluoromethyl)-phenyl amide of formula 4b (12.5 g 1.0 eq) in chlorobenzene (150 g) at 70-75° C., 4-(4-aminophenoxy)-N-methyl-2-pyridinecarboxamide (10 g) was added and the mixture was kept under stirring at 70-75° C. for 15 minutes until a precipitate formed. The suspension was then cooled to 10° C. and the precipitate was filtered off, washed with chlorobenzene, water and dried at 60° C. under vacuum overnight. Sorafenib was obtained as a colorless powder (18.5 g, 95% yield, 99.7% purity).

### Example 5

### Preparation of Sorafenib Tosylate as Described in Method 5c of WO 2006/034796

4-{4-[({[4-Chloro-3-(trifluoromethyl)phenyl]amino}carbonyl)amino]phenoxy}-N-methylpyridine-2-carboxamide (50 g, 0.1076 mol) is suspended in ethyl acetate (500 g) and water (10 g). The mixture is heated to 69° C. within 0.5 h, and a filtered solution of p-toluenesulfonic acid monohydrate (3.26 g, 0.017 mol) in a mixture of water (0.65 g) and ethyl acetate (7.2 g) is added. After filtration a filtered solution of p-toluenesulfonic acid monohydrate (22 g, 0.11 mol) in a mixture of ethyl acetate (48 g) and water (4.34 g) is added. The mixture is cooled to 23° C. within 2 h. The product is filtered off, washed twice with ethyl acetate (92.5 g each time) and dried under reduced pressure. The title compound (65.5 g, 96% of theory) is obtained as colorless to slightly brownish crystals.

### Example 6

### Preparation of Sorafenib Base According to WO 2006/034796

4-(4-Aminophenoxy)-N-methyl-2-pyridinecarboxamide (5.23 g, 21.5 mmol) was suspended in ethyl acetate (14.6 g) and the suspension was heated to 40° C. Then 4-chloro-3-trifluoromethylphenylisocyanate (5 g, 22.6 mmol), dissolved in ethyl acetate (5.8 g) was added to such a degree that the temperature is kept below 60° C. After cooling to 20° C. within 1 hour, the mixture was stirred for a further 30 min and the product was filtered off. After washing with ethyl acetate (3 g), the product was dried under reduced pressure at 50° C. The title product was obtained as a brownish powder (9.45 g, 94.5% yield).

We claim:

1. A compound of the following formula: 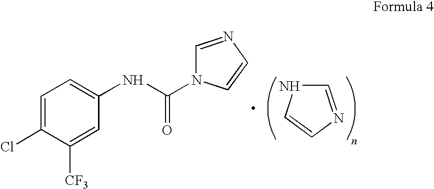
wherein n is either 0 or 1.

2. The compound of claim 1, wherein the compound is isolated.

3. The compound of claim 1, wherein n=1, corresponding to an imidazole complex of imidazole-1-carboxylic acid 4-chloro-3-(trifluoromethyl)phenyl amide having the following formula:

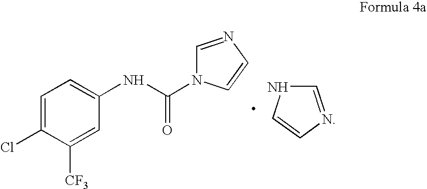

4. The compound of claim 3 characterized by a ¹H-NMR (300 MHz) spectrum in CDCl₃ having chemical shifts at 7.07, 7.12, 7.46, 7.67, 7.77, 7.83, 7.92, 8.45 and 10.7.

5. The compound of claim 1, wherein n=0, corresponding to imidazole-1-carboxylic acid 4-chloro-3-(trifluoromethyl)phenyl amide having the following formula:

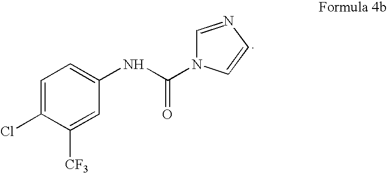

6. The compound of claim 5, characterized by data selected from the group consisting of: a ¹H-NMR (300 MHz) spectrum in CDCl₃ having chemical shifts at about 7.913, 7.836, 7.480, 8.418, 7.725 and 7.097; a mass spectrum of MS-ESI [M+H]⁺=m/z 290/292; and combinations thereof.

7. A process for preparing Sorafenib base comprising reacting the compound of formula 4 of the following formula: 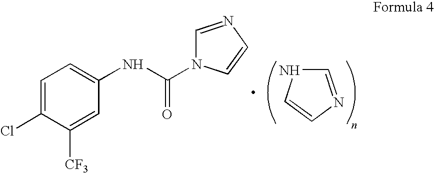
with 4-(2-N-methylcarbamoyl)-4-pyridyloxy)aniline of formula 2: 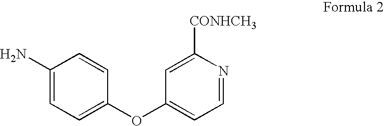
wherein n is either 1 or 0.

8. The process of claim 7, wherein said process comprises a) dissolving the compound of formula 4 to obtain a solution; b) adding the compound of formula 2 to obtain a mixture, and c) precipitating Sorafenib base.

9. The process of claim 8, wherein the solvent in the solution of step (a) is selected from the group consisting of: halogenated hydrocarbons, esters, and mixtures thereof.

10. The process of claim 9, wherein the solvent is selected from the group consisting of: C₁-C₆ halogenated hydrocarbon, C₂-C₆ ester, and mixtures thereof.

11. The process of claim 10, wherein the solvent is selected from the group consisting of: 1,2-dichloroethane, chlorobenzene, chloroform, ethyl acetate, ethyl formate, ethyl propionate, methyl acetate, and mixtures thereof.

12. The process of claim 7, further comprising recovering Sorafenib base.

13. The process of claim 7, further comprising converting Sorafenib base to Sorafenib salt.

14. The process of claim 13, wherein the salt is p-toluenesulfonic acid (“PTSA”) salt.

15. The process of claim 7, wherein:
(a) n is 1, and the compound of formula 4 is prepared by a process comprising reacting 4-chloro-3-(trifluoromethyl)aniline of formula 3 and carbonyldiimidazole (CDI); or
(b) n is 0, and the compound of formula 4 is prepared by a process comprising reacting 4-chloro-3-(trifluoromethyl)phenyl isocyanate of formula 1 and imidazole of formula 6.

16. A process for preparing an imidazole complex of imidazole-1-carboxylic acid 4-chloro-3-(trifluoromethyl)phenyl amide of the following formula: 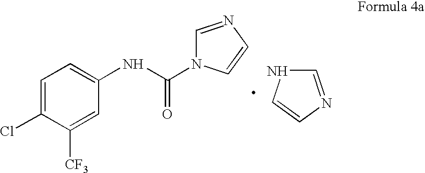
comprising reacting 4-chloro-3-(trifluoromethyl)aniline of the following formula: 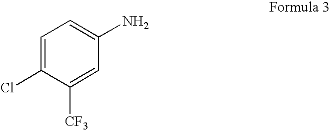
with carbonyldiimidazole (CDI) of formula 5 having the following structure;

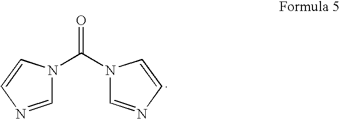

17. The process of claim 16, comprising (a) providing a solution of 4-chloro-3-(trifluoromethyl)aniline of formula 3; and (b) adding carbonyldiimidazole (CDI) of formula 5 to obtain a suspension comprising the imidazole complex of imidazole-1-carboxylic acid 4-chloro-3-(trifluoromethyl)phenyl amide of formula 4a.

18. The process of claim 17, wherein the solvent in the solution of step (a) is halogenated hydrocarbon.

19. The process of claim 18, wherein the halogenated hydrocarbon is a C₁-C₆ halogenated hydrocarbon.

20. The process of claim 19, wherein the C₁-C₆ halogenated hydrocarbon is dichloromethane, chlorobenzene or chloroform.

21. The process of claim 16, further comprising recovering the imidazole complex of imidazole-1-carboxylic acid 4-chloro-3-(trifluoromethyl)phenyl amide of formula 4a.

22. A process for preparing imidazole-1-carboxylic acid 4-chloro-3-(trifluoromethyl)phenyl amide of the following formula: 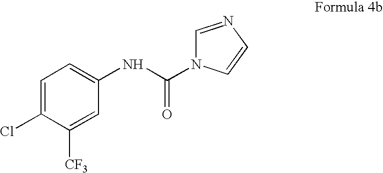
comprising reacting 4-chloro-3-(trifluoromethyl)phenyl isocyanate of the following formula: 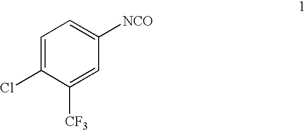
with imidazole or a salt thereof of formula 6: 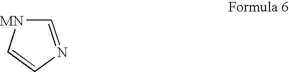
wherein M is hydrogen, sodium or potassium.

23. The process of claim 22, comprising (a) providing a solution of imidazole of formula 6, and (b) adding 4-chloro-3-(trifluoromethyl)phenyl isocyanate of formula 1 to obtain a suspension comprising imidazole-1-carboxylic acid 4-chloro-3-(trifluoromethyl)phenyl amide of formula 4b.

24. The process of claim 23, wherein the solvent in the solution of step (a) is a halogenated hydrocarbon.

25. The process of claim 24, wherein the halogenated hydrocarbon is a C₁-C₆ halogenated hydrocarbon.

26. The process of claim 24, wherein the C₁-C₆ halogenated hydrocarbon is chloroform.

27. The process of claim 22, further comprising recovering imidazole-1-carboxylic acid 4-chloro-3-(trifluoromethyl)phenyl amide of formula 4b.

28. A process for preparing Sorafenib or a salt thereof of the following formula: 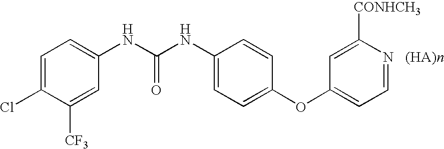
comprising preparing the compounds of formula 4a or 4b according claims 16 or 22 and converting either of them to Sorafenib or a salt thereof; wherein n is either 0 or 1, and HA is an acid.

29. The process of claim 28, wherein the acid is p-toluenesulfonic acid (“PTSA”).
